(19) United States
(12) Patent Application Publication
(10) Pub. No.: US 2026/0159997 A1
(43) Pub. Date: Jun. 11, 2026

(54) THERMOREGULATING THERAPEUTIC YARN, SMART FABRIC INCLUDING THE YARN AND ARTICLES MADE THEREFROM

(71) Applicant: Supreme Corporation, Conover, NC (US)

(72) Inventors: Rakkiyappan CHANDRAN, Hickory, NC (US); Matthew KOLMES, Hickory, NC (US)

(73) Assignee: Supreme Corporation, Conover, NC (US)

(21) Appl. No.: 18/650,658

(22) PCT Filed: Feb. 16, 2024
(86) PCT No.: PCT/US2024/016108

§ 371 Date: Apr. 30, 2024

Related U.S. Application Data
(60) Provisional application No. 63/485,381, filed on Feb. 16, 2023.

(51) Int. Cl. D02G 3/36; A41B 1/08; A41B 9/00; A41B 11/00; A47G 9/02; A61F 13/00; D02G 3/04; D06M 11/79; D06M 11/84; D06M 101/40

(52) U.S. Cl. CPC D02G 3/36; A61F 13/00063; D02G 3/04; D06M 11/79; D06M 11/84; A41B 1/08; A41B 9/00; A41B 11/00; A41B 2500/10; A41B 2500/20; A47G 9/0238; D06M 2101/40; D10B 2101/12; D10B 2401/04; D10B 2501/04

(57) ABSTRACT

The present invention provides a thermoregulating therapeutic yarn. The yarn includes a first plurality of fibers, a second plurality of fibers, and nanoparticles. The second plurality of fibers comprises a carbon material. The first and second pluralities of fibers together form a yarn structure. The nanoparticles are adhered to outer surfaces of the first plurality of fibers, second plurality of fibers, or the yarn structure and are capable of upconverting incident wavelengths.

### CROSS REFERENCE TO RELATED APPLICATIONS

This application is related to, and claims priority to, U.S. Provisional Application Ser. No. 63/485,381, filed Feb. 16, 2023, pending, the entire contents of which are incorporated herein by reference.

### FIELD OF THE INVENTION

The present invention relates to a thermoregulating therapeutic smart fabric that has a biological effect on the wearer's body, the yarn used to prepare the fabric, and articles made from the fabric.

### DESCRIPTION OF THE RELATED ART

Thermoregulation is a problem that affects human beings in a variety of occupations and environments around the world. According to Joseph Rampulla, MS, APRN, BC “Heat illness is generally underreported, and the true incidence is unknown. Death rates from other causes (e.g. cardiovascular, respiratory) increase during heat waves but are generally not reflected in the morbidity and mortality statistics related to heat illness. Nonetheless, heat waves account for more deaths than all other natural disasters combined in the USA.” From police and paramedics to construction workers and laborers, or service men and women engaging in strenuous exercise and physical training, hot and humid conditions degrade performance abilities and lead to injuries, fainting, and other more serious medical conditions. Contrarily, when human beings are subjected to cold temperatures for prolonged periods of time, poor thermoregulation can cause the body to slow down and even cause frostbite. Nevertheless, heat exhaustion and prolonged exposure to cold temperatures can also hinder the body's ability to recover from pre-existing injuries.

Most of the available products today for body cooling use lightweight polyester material with a finish that wicks moisture to create a lightweight fabric that will wick moisture away from the body. This creates a material that has some properties that will keep you cool (wicking pulls sweat away from your skin). While polyester is a durable fiber, it does best to insulate your body in cold temperatures. Fabrics and shirts made from polyester, with a wicking finish are readily available from every major sportswear company. The effectiveness of these polyester wicking fabrics at keeping your body comfortable in heat and humidity is minimal and quickly dissipates as the fiber becomes overloaded with body heat and perspiration.

Most of the available products today for body heating use heavier, tighter, and/or thicker materials to insulate the body. These bulky materials can hinder the wearer's mobility and thus, hinder performance during physical activities. Additionally, if the wearer becomes too hot, they must remove the products, and then, put the products back on when they become cold again. Such manual regulation of temperature in cold conditions takes is time-consuming and a hassle while performing physical activities. The bulky products can also be difficult to wash in convention household washing machines.

Accordingly, a fabric is needed that will provide more consistent and efficient thermoregulation of the wearer for both cold and hot conditions.

### SUMMARY OF THE INVENTION

Accordingly, one object of the present invention is to provide a thermoregulating therapeutic yarn that can be used to prepare a thermoregulating therapeutic smart fabric.

Another object of the present invention is to provide a thermoregulating therapeutic smart fabric comprising the yarn, where the fabric mimics thermoregulation in the body and improves cooling and/or heating of a system covered by the thermoregulating therapeutic smart fabric.

A further object of the invention is to provide a thermoregulating therapeutic smart fabric that provides a thermoregulation effect and also provides other therapeutic effects to improve vital signs of the wearer's body.

Another object of the invention is provide a garment formed from the thermoregulating therapeutic smart fabric, which is designed to be washable and to be worn as a base layer to constantly provide thermoregulation and therapeutic benefits to the wearer.

Yet another object of the invention is to provide a thermoregulating therapeutic smart fabric designed to provide healing effects to improve performance of the wearer and to also prolong the time necessary for the wearer to seek medical attention.

These and other objects of the present invention, alone or in combinations thereof, have been satisfied by the discovery of a thermoregulating therapeutic yarn and smart fabric prepared therefrom, comprising nanoparticles attached on outer surfaces of a thermoregulating therapeutic yarn. The thermoregulating therapeutic yarn includes a first plurality of fibers and a second plurality of fibers, wherein the second plurality of fibers comprises a carbon material. The nanoparticles contain various dopants that can upconvert different wavelengths, thereby providing the thermoregulating and therapeutic effects of the overall fabric.

### DETAILED DESCRIPTION OF THE PREFERRED EMBODIMENTS

The term “fiber” as used herein refers to a fundamental component used in the assembly of yarns and fabrics. Generally, a fiber is a component which has a length dimension which is much greater than its diameter or width. This term includes ribbon, strip, staple, and other forms of chopped, cut or discontinuous fiber and the like having a regular or irregular cross section. “Fiber” also includes a plurality of any one of the above or a combination of the above.

As used herein, the term “high performance fiber” means that class of synthetic or natural non-glass fibers having high values of tenacity greater than 10 g/denier, such that they lend themselves for applications where high abrasion and/or cut resistance is important. Typically, high performance fibers have a very high degree of molecular orientation and crystallinity in the final fiber structure.

The term “filament” as used herein refers to a fiber of indefinite or extreme length such as found naturally in silk. This term also refers to manufactured fibers produced by, among other things, extrusion processes. Individual filaments making up a fiber may have any one of a variety of cross sections to include round, serrated or crenular, bean-shaped or others.

The term “yarn” as used herein refers to a continuous strand of textile fibers, filaments or material in a form suitable for knitting, weaving, or otherwise intertwining to form a textile fabric. Yarn can occur in a variety of forms to include a spun yarn consisting of staple fibers usually bound together by twist; a multi filament yarn consisting of many continuous filaments or strands; or a mono filament yarn which consists of a single strand. A “blended yarn” as used herein refers to a yarn that comprises an intimate blend of at least two different types of fibers.

The term “composite yarn” refers to a yarn prepared from two or more yarns, which can be the same or different. Composite yarn can occur in a variety of forms wherein the two or more yarns are in differing orientations relative to one another. The two or more yarns can, for example, be parallel, wrapped one around the other(s), twisted together, or combinations of any or all of these, as well as other orientations, depending on the properties of the composite yarn desired. Examples of such composite yarns are provided in U.S. Pat. Nos. 4,777,789, 4,838,017, 4,936,085, 5,177,948, 5,628,172, 5,632,137, 5,644,907, 5,655,358, 5,845,476, 6,212,914, 6,230,524, 6,341,483, 6,349,531, 6,363,703, 6,367,290, and 6,381,940 (collectively, the “Kolmes patents”), the contents of each of which are hereby incorporated by reference.

For convenience, the term “yarn component” as used herein, encompasses fiber, monofilament, multifilament and yarn.

The present invention relates to a thermoregulating therapeutic yarn and a thermoregulating therapeutic smart fabric prepared therefrom, wherein the thermoregulating therapeutic yarn comprises a first plurality of fibers and a second plurality of fibers wherein nanoparticles are attached on outer surfaces of the thermoregulating therapeutic yarn that provide thermoregulating and therapeutic effects to a wearer's body. In some embodiments, the first plurality of fibers comprises, for example, a natural material (e.g., cotton, silk, etc.) or a synthetic material (e.g., nylon, polyester, Lyocell, a para-aramid like Kevlar, etc.). In some embodiments, the first plurality of fibers comprises a single type of fiber, whereas in other embodiments, the first plurality of fibers comprises a blend of multiple natural and/or synthetic fibers. Additionally, the first plurality of fibers may be chosen based on desired properties of the overall thermoregulating therapeutic yarn such as elasticity, high-performance, hydrophobicity, weight, flame-resistance, or some other desired property. Examples of such fibers will be described further herein.

The second plurality of fibers comprises a carbon material, which may be in the form of, for example, carbon fibers, graphene, graphene fibers, graphite, carbon nanotubes, C60 fullerene, a combination thereof, or the like. Carbon is a durable and long-lasting material as it does not suffer from fatigue, rotting, corrosion, or degradation in poor conditions. In some embodiments, carbon fibers or graphene fibers are preferred for their size and ease of forming into a fiber and yarn. The carbon material provides favorable heat dissipation properties to thermoregulate a wearer's body of the overall thermoregulating therapeutic smart fabric while also being able to withstand wear and tear. For example, the activated carbon material can absorb infrared radiation from the human body efficiently. Additionally, the carbon material can further function as a photosensitizer, which transfers energy from received light to other particles nearby in the thermoregulating therapeutic yarn, such as the nanoparticles as will be described further herein. Because the carbon material of the second plurality of fibers is arranged at least on outer surfaces of the thermoregulating therapeutic yarn, the carbon material can more efficiently receive heat/received light from the body and/or environment, and thus, more efficiently create the thermoregulation effects of the invention.

It will be appreciated that the structure of the first and second plurality of fibers within the yarn can be one of many structures as long as at least the second plurality of fibers, which contains carbon, is arranged on outer surfaces of the thermoregulating therapeutic yarn. For example, in some embodiments, the thermoregulating therapeutic yarn may be a composite yarn, wherein each of the first plurality of fibers and the second plurality of fibers are yarn. In some such embodiments, the composite yarn comprises a core yarn. The composite yarn may further comprise one or more cover yarns wrapped around the core yarn. The first plurality of fibers may be the core yarn and/or the one or more cover yarns. Similarly, the second plurality of fibers may be the core yarn and/or the one or more cover yarns. Further, the one or more cover yarns may be wrapped around the core layer according to any wrapping pattern. In other embodiments, the yarn may be blended, wherein the first and second plurality of fibers are comingled to form the blended yarn. In some embodiments, several blended yarns comprising the first and second plurality of fibers may be wrapped together forming a composite yarn. The thermoregulating therapeutic yarn can be any desired denier, preferably from 10 to 325, more preferably from 50 to 250, most preferably from 100 to 220. The disclosed yarn is not limited to the aforementioned structures and thus, other yarn structures are also within the scope of this disclosure.

In some embodiments, about 5 weight percent (wt %) to about 40 wt % of the thermoregulating therapeutic yarn comprises carbon material. A higher content of carbon material within the yarn increases the thermoregulation effects in the thermoregulating therapeutic yarn. In some other embodiments, about 10 wt % to about 35 wt % of the thermoregulating therapeutic yarn comprises carbon material. More preferably, in some embodiments, about 15 wt % to about 25 wt % of the thermoregulating therapeutic yarn comprises a carbon material, which provides efficient thermoregulation effects at a reasonable cost. For example, when less than 15 wt % of the thermoregulating therapeutic yarn comprises a carbon material, the thermoregulating effects provided by the carbon material may still be present but at a lower efficiency. In some embodiments, the nanoparticles make up about 0.5 wt % to about 3.5 wt % of the thermoregulating therapeutic yarn. More preferably, in some embodiments, the nanoparticles make up about 1 wt % to about 2 wt % of the thermoregulating therapeutic yarn. Because the nanoparticles have a very large surface area to volume ratio, coating about at most 2 wt % of the thermoregulating therapeutic yarn with the nanoparticles is sufficient to achieve a yarn having thermoregulating and therapeutic effects.

The nanoparticles have wavelength upconverting properties, meaning that the nanoparticles can change the wavelength of received light (in this respect, in the context of the present invention, the term “upconverting” refers to converting a lower energy radiation to a higher energy radiation (i.e. converting a longer wavelength radiation to a shorter wavelength radiation). In some embodiments, the nanoparticles comprise materials configured to convert a received higher wavelength like infrared light to a lower wavelength like visible light. Thus, the nanoparticles absorb light having a first energy and first wavelength and emit light having a second energy that is higher than the first energy and a second wavelength that is smaller than the first wavelength. In some embodiments, the carbon from the second fibers in the thermoregulating therapeutic yarn absorb heat (e.g., infrared radiation) from the wearer's body, which is then converted to visible light using the upconverting nanoparticles.

In some embodiments, the nanoparticles comprise metal-oxide coated upconverting nanoparticles with various dopants to achieve the upconverting properties. The type and concentration of dopants in the nanoparticles can be tuned to achieve a desired visible light wavelength after upconverting. For example, in some embodiments, the nanoparticles comprise mesoporous SiO₂ coated upconverting nanoparticles, SiO₂—COOH modified upconverting nanoparticles, SiO₂ dense upconverting nanoparticles such as DIAGNANO Dense Silica Upconverting Nanoparticles sold by CD Bioparticles, or some other suitable nanoparticle that provides upconverting effects. As will be discussed further herein, the upconverting effects of the nanoparticle provide thermoregulating and therapeutic effects. The nanoparticles are adhered to the surface of the thermoregulating therapeutic yarn such that the thermoregulating therapeutic yarn has the wavelength upconverting properties. In one embodiment, the nanoparticles are adhered to the first and/or second pluralities of fibers, and then, the first and second pluralities of fibers are assembled together in a desired yarn structure to form a thermoregulating therapeutic yarn. In another embodiments, the first and second pluralities of fibers are first assembled together in a desired yarn structure, and then, the nanoparticles are adhered to the desired yarn structure to form the thermoregulating therapeutic yarn.

To adhere the nanoparticles to the surface of the overall thermoregulating therapeutic yarn or to the first and/or second pluralities of fibers of the thermoregulating therapeutic yarn, a bonding agent and optionally a lubricant is used. The bonding agent and lubricant can be applied in any conventional manner, including but not limited to spraying on the yarn, kiss-roll, or dipping the yarn into a bath containing the bonding agent or lubricant, either neat or as a solution in a suitable organic or aqueous solvent. The preferred lubricant is a silicone with paraffin added. Additional lubricants which have been found to be satisfactory are—RAYOLAN 1813, Boehme FILATEX, or KL 400 (Kelmar).

After the optional step of lubrication, the overall thermoregulating therapeutic yarn or the first and/or second pluralities of fibers thereof are then treated with at least one suitable bonding agent, including but not limited to at least one member selected from the group consisting of polyurethanes, polyacrylics, nylons, some other suitable polymeric substance, and other conventional fiber bonding compositions. The nanoparticles are mixed in the bonding agent to form a bonding agent mixture. The bonding agent mixture may be applied to the thermoregulating therapeutic yarn or fibers thereof through a variety of methods such as, for example, dip-coating, roll-coating, spraying, or the like. Once applied, the bonding agent is permitted to cure to provide sufficient bonding of the fibers of the thermoregulating therapeutic yarn, thereby securing the nanoparticles to the surfaces of the thermoregulating therapeutic yarn. In the present invention, the term “cure” or “curing” includes, but is not limited to, drying of the bonding agent, polymerizing of the bonding agent, crosslinking of the bonding agent either with itself or the surface of the fibers to which it is applied, for example. Thus, in a preferred embodiment, the thermoregulating therapeutic yarn comprises nanoparticles suspended in a cured bonding agent that is adhered to outer surfaces of the thermoregulating therapeutic yarn. In some embodiments, a lubricant is also arranged between the outer surfaces of the thermoregulating therapeutic yarn and the cured bonding agent.

In the present invention, the thermoregulating therapeutic yarn may be woven into a fabric that can be formed into articles, preferably into an article selected from the group consisting of garments, bed sheets, pillowcases, and bandages. In a particularly preferred embodiment, a garment formed from the present invention yarn is formed by shaped knitting (knitting that uses dropped stitches in order to generate a garment in a particular shape, with minimal seams in construction). In some embodiments, the thermoregulating therapeutic yarn is first woven into a thermoregulating therapeutic smart fabric, and then the thermoregulating therapeutic smart fabric is assembled into the article. In other embodiments, the thermoregulating therapeutic yarn is woven directly into a desired article. It will be appreciated that other techniques to transform the thermoregulating therapeutic yarn into a fabric and garment are within the scope of this disclosure.

When the article is a garment, it is preferably a member selected from the group consisting of shirts, undergarments, socks, leggings, biking pants/shorts and tights. When the garment is a shirt, it can be either short-sleeved or long-sleeved, and when the garment is biking pants it can be either long legged or shorts. More preferably, in some embodiments, the garment is designed to be worn under the wearer's typical clothing. In some such embodiments, the garment is designed to be thin and conform to the wearer's body such that the garment does not interfere with the fit and comfort of the wearer's typical clothing overlying the garment such that the thermoregulating therapeutic yarn in the garment is closest to the wearer's skin to provide thermoregulating and therapeutic effects. Thus, in some such embodiments, the thermoregulating therapeutic smart fabric may be formed into a garment such as a pair of elastic, spandex-like leggings or pants and a spandex-like long-sleeved shirt. Additionally, the thermoregulating therapeutic smart fabric is designed to be survive washing without losing its thermoregulating and therapeutic properties. In some embodiments, the thermoregulating therapeutic smart fabric can survive more than 5 wash cycles, preferably more than 10 wash cycles, more preferably more than 20 wash cycles, and most preferably more than 30 wash cycles. In some embodiments, a suitable bonding agent can withstand the wash cycles such that the nanoparticles remain suspended in the bonding agent and adhered to the thermoregulating therapeutic yarn even after the many wash cycles.

As a garment, the thermoregulating therapeutic yarn provides both thermoregulating and therapeutic effects to the wearer of the garment to improve vital signs of the wearer's body. The carbon from the second fibers and the upconverting nanoparticles in the thermoregulating therapeutic yarn are configured to react with radiation emitted from the wearer's body and radiation emitted elsewhere in the body's environment to convert the radiation's wavelength to a different/lower wavelength to create a heating effect. The carbon from the second fibers also delay and dissipate heat energy to create a cooling effect. In turn, the thermoregulating therapeutic yarn continuously regulates its surrounding radiation to provide cooling or heating effects to the body for thermoregulation of the body. The upconverting nanoparticles may also emit wavelengths that kill bacteria, thereby making the thermoregulating therapeutic yarn anti-bacterial. Further, the carbon material of the second plurality of fibers of the thermoregulating therapeutic yarn are configured to distribute the received radiation and heat energy throughout the garment to distribute the cooling or heat effects to the body covered by the garment. Because the carbon material of the second plurality of fibers of the thermoregulating therapeutic yarn are arranged at least on outer surfaces of the thermoregulating therapeutic yarn, the distribution of received radiation and heat energy is effective and efficient.

In some embodiments, the thermoregulating therapeutic yarn further comprises dye molecules adhered to the first and/or second pluralities of fibers. The dye molecules may be adhered to the first and/or second pluralities of fibers via the bonding agent or the lubricant, for example. Thus, in some embodiments, the bonding agent mixture may also comprise the dye molecules. In other embodiments, the dye molecules may be adhered to the first and/or second pluralities of fibers in a different process than the bonding agent and lubricant processes. When the thermoregulating therapeutic yarn is in use, the visible light emitted by the nanoparticles may activate the dye molecules coated on the yarn. The activated dye molecules provide therapeutic effects to the wearer by producing a reactive oxygen species. The dye molecules are adhered at least to the fibers that comprise the nanoparticles such that the nanoparticles can efficiently activate the dye molecules. Thus, in some embodiments, the first plurality of fibers comprise the nanoparticles and the dye molecules and/or the second plurality of fibers comprise nanoparticles and the dye molecules. In some embodiments, the dye molecules comprise Rose Bengal or some other suitable dye molecule capable of absorbing a wavelength emitted by the nanoparticles and emitting at a wavelength configured to provide the desired therapeutic effect.

When worn over a period of days and weeks, the thermoregulating therapeutic garment decreases cortisol levels in the blood and increases nitric oxide in the wearer's body. Additionally, an increase of nitric oxide in one's body causes reduced inflammation, muscle spasm relief, detoxification, increased blood oxygen level, increased blood flow and micro-circulation, improved oxygen delivery to the brain, improved sleep quality, pain relief, enhanced white blood cell function for an improved immune system, accelerated wound healing, restless leg syndrome relief, improved sense of calm, improvement in men and women's reproductive health, reduced anxiety and PTSD, and improved heart rate variability. The improved blood flow supports of all body systems including the cardiovascular system, the nervous system, the immune system, the digestive system, and the reproductive system.

By improving the body's vital signs as mentioned above, the wearer's athletic performance is improved. Thus, athletes, blue-collar workers, or other laborers of the thermoregulating therapeutic garments may have more stamina to complete tasks. The wound and inflammation healing effects of the thermoregulating therapeutic garments may also accelerate the wearer's recovery and/or also prolong the time necessary for a wearer to seek medical attention for injuries. Thus, these thermoregulating therapeutic garments would be useful for those in the military, shipmates, or anyone who is in a remote location with limited access to medical assistance. For example, a person stranded in sea water would have an improved chance of survival while waiting for rescue thanks to the thermoregulating and therapeutic effects of the invention. The thermoregulating therapeutic smart fabric also does not comprise metal, which allows it to function in water without corrosion. Additionally, by converting emitted radiation from the wearer's body, the thermoregulating therapeutic garment may also reduce or block the wearer's infrared heat signature. This may also be useful in undercover military missions such that the wearer's cannot be identified by enemies via infrared detection.

It will be appreciated that the thermoregulating therapeutic smart fabric may also be used to cover other structures than the body. For example, the thermoregulating therapeutic smart fabric may be used to cover a battery or some other object at least for thermoregulation. The thermoregulating therapeutic smart fabric does not need to be plugged in to function and thus, provides a sustainable method of thermoregulation.

Most preferably, no other type of yarn is used in the thermoregulating therapeutic fabric and articles made therefrom of the present invention besides the thermoregulating therapeutic yarn, with the exception of thread used to stitch parts of the fabric together at the seams. However, it will be appreciated that depending on the application, additional yarns may be needed to provide the desired garment properties. If the one or more additional yarns are included, the yarn can be any of a variety of types of yarn.

For example, the first plurality of fibers, the second plurality of fibers, or additional yarns in the thermoregulating therapeutic yarn may comprise an elastomeric fiber. An elastomer is a natural or synthetic polymer that, at room temperature, can be stretched and expanded to typically twice its original length. After removal of the tensile load it will immediately return to its original length. Along with spandex, rubber and anidex (no longer produced in the United States) are considered elastomeric fibers. Spun from a block copolymer, spandex fibers exploit the high crystallinity and hardness of polyurethane segments, yet remain “rubbery” due to alternating segments of polyethylene glycol. Suitable elastomeric fibers include, but are not limited to, fibers made from copolymers having both rigid and flexible segments in the polymer chains, such as, for example, block copolymers of polyurethane and polyethylene glycol. Particularly suitable elastomeric fibers include, but are not limited to, Spandex, such as LYCRA (produced by United Yarn Products), ELASPAN (produced by Invista), DORLASTAN (produced by Bayer), CLEAR SPAN (produced by Radici) and LINEL (produced by Fillattice).

Elastomeric fibers can have one or more of the following materials properties: can be stretched over 500% without breaking; able to be stretched repetitively and still recover original length; lightweight; abrasion resistant; poor strength, but stronger and more durable than rubber; soft, smooth, and supple; resistant to body oils, perspiration, lotions, and detergents; no static or pilling problem; very comfortable; and easily dyed.

The first plurality of fibers can be any desired denier, preferably from 10 to 210, more preferably from 15 to 150, most preferably from 20 to 75. The elastomeric fiber can be used alone or combined with one or more other fibers of any desired type to form the first plurality of fibers, so long as the combination retains its elastomeric properties. If combined with one or more other fibers, the elastomeric fiber and other fibers are preferably blended to form the first plurality of fibers.

In some embodiments, the second plurality of fibers may be wrapped around the first plurality of fibers to form a composite yarn structure, wherein the core yarn is the first plurality of fibers. The composite yarn structure would be elastic due to the elastomeric core yarn comprising the first plurality of fibers. Elastomeric yarn containing composite yarns are further described in U.S. Pat. Nos. 5,568,657 and 5,442,815, the contents of which are incorporated herein by reference. Elastomeric yarn containing composite yarns having wicking properties are described in U.S. Provisional Application Ser. No. 61/020,790, filed Jan. 14, 2008, the contents of which are hereby incorporated by reference.

As another example, if high strength, tenacity and/or cut resistance are desired for the thermoregulating therapeutic yarn, the first plurality of fibers, the second plurality of fibers, or additional yarns in the thermoregulating therapeutic yarn may comprise small amounts of a high performance fiber in the present invention fabric. The high performance fiber can be any desired high performance fiber. If hydrophobicity is also desired, the high performance fiber preferably comprises a high molecular weight polyolefin, preferably high molecular weight polyethylene or high molecular weight polypropylene.

U.S. Pat. No. 4,457,985, hereby incorporated by reference, generally discusses high molecular weight polyethylene and polypropylene fibers. In the case of polyethylene, suitable fibers are those of molecular weight of at least 150,000, preferably at least 400,000, more preferably at least one million and most preferably between two million and five million. Such extended chain polyethylene (ECPE) fibers may be grown in solution as described in U.S. Pat. No. 4,137,394 or U.S. Pat. No. 4,356,138, hereby incorporated by reference, or may be a filament spun from a solution to form a gel structure, as described in German Off. 3 004 699 and GB 2 051 667, and especially described in U.S. Pat. No. 4,551,296, hereby incorporated by reference. As used herein, the term polyethylene preferably means a predominantly linear polyethylene material that may contain minor amounts of chain branching or comonomers not exceeding 5 modifying units per 100 main chain carbon atoms, and that may also contain admixed therewith not more than about 50 weight percent of one or more polymeric additives such as alkene-1-polymers, in particular low density polyethylene, polypropylene or polybutylene, or copolymers containing mono-olefins as primary monomers. Depending upon the formation technique, the draw ratio and temperatures, and other conditions, a variety of properties can be imparted to these fibers. The tenacity of the fibers should preferably be at least 15 grams per denier (g/d), more preferably at least 20 g/d, even more preferably at least 25 g/d and most preferably at least 28 g/d. Similarly, the tensile modulus of the filaments, as measured by an Instron tensile testing machine, is preferably at least 300 g/d, more preferably at least 500 g/d and still more preferably at least 1,000 g/d and most preferably at least 1,200 g/d. These highest values for tensile modulus and tenacity are generally obtainable only by employing solution grown or gel fiber processes. For example, high molecular weight polyethylene filaments produced commercially by Honeywell Corp. under the trade name SPECTRA or by DSM under the trade name DYNEEMA and having moderately high moduli and tenacity are particularly useful.

Similarly, highly oriented polypropylene of molecular weight at least 200,000, preferably at least one million and more preferably at least two million, may be used. Such high molecular weight polypropylene may be formed into reasonably well oriented fibers by techniques described in the various references referred to above, and especially by the technique of U.S. Pat. Nos. 4,663,101 and 4,784,820, hereby incorporated by reference, and U.S. patent application Ser. No. 069,684, filed Jul. 6, 1987 (see published application WO 89 00213). Since polypropylene is a much less crystalline material than polyethylene and contains pendant methyl groups, tenacity values achievable with polypropylene are generally substantially lower than the corresponding values for polyethylene. Accordingly, a suitable tenacity is at least about 8 g/d, with a preferred tenacity being at least about 11 g/d. The tensile modulus for polypropylene is at least about 160 g/d, preferably at least about 200 g/d. The high performance yarn can be any desired denier, preferably from 10 to 325, more preferably from 50 to 250, most preferably from 100 to 220.

The present invention fabric provides significantly more thermoregulating and therapeutic effects to the wearer to improve the wearer's vital signs to prevent or prolong the need for medical assistance.

The following presents a non-limiting listing of embodiments of the present invention:

Embodiment 1. A thermoregulating therapeutic yarn comprising:
a first plurality of fibers;
a second plurality of fibers comprising a carbon material, wherein the first and second pluralities of fibers together form a yarn structure; and
nanoparticles adhered to outer surfaces of the first plurality of fibers, second plurality of fibers, or the yarn structure formed therefrom, wherein the nanoparticles are capable of upconverting incident wavelengths.

Embodiment 2. The thermoregulating therapeutic yarn of Embodiment 1, wherein about 15 wt % to about 25 wt % of the thermoregulating therapeutic yarn comprises the carbon material.

Embodiment 3. The thermoregulating therapeutic yarn of one of Embodiments 1 or 2, wherein the nanoparticles are present in an amount of from about 1 wt % to about 2 wt % of the thermoregulating therapeutic yarn.

Embodiment 4. The thermoregulating therapeutic yarn of any one of Embodiments 1 to 3, further comprising a bonding agent.

Embodiment 5. The thermoregulating therapeutic yarn of Embodiment 4, wherein the nanoparticles are suspended within the bonding agent.

Embodiment 6. The thermoregulating therapeutic yarn of one of Embodiments 4 or 5, further comprising a lubricant, wherein the lubricant is arranged between the bonding agent and the first and second pluralities of fibers.

Embodiment 7. The thermoregulating therapeutic yarn of any one of Embodiments 1 to 6, wherein the second plurality of fibers is arranged on the outer surfaces of the yarn structure.

Embodiment 8. The thermoregulating therapeutic yarn of any one of Embodiments 1 to 7, wherein the yarn structure is a composite yarn, the first plurality of fibers is a core yarn of the composite yarn, and the second plurality of fibers is a cover yarn of the composite yarn.

Embodiment 9. The thermoregulating therapeutic yarn of any one of Embodiments 1 to 7, wherein the yarn structure is a blended yard, wherein the first and second pluralities of fibers are comingled.

Embodiment 10. The thermoregulating therapeutic yarn of any one of Embodiments 1 to 9, wherein the nanoparticles comprise silica.

Embodiment 11. The thermoregulating therapeutic yarn of any one of Embodiments 1 to 10, wherein the carbon material comprises carbon fibers.

Embodiment 12. The thermoregulating therapeutic yarn of any one of Embodiments 1 to 11, further comprising dye molecules configured to produce reactive oxygen species when activated by visible light.

Embodiment 13. An article prepared from the thermoregulating therapeutic yarn of any one of Embodiments 1 to 12.

Embodiment 14. The article of Embodiment 13, wherein the article is a member selected from bed sheets, pillowcases, and bandages.

Embodiment 15. The article of Embodiment 13, wherein the article is a garment.

Embodiment 16. The article of Embodiment 15, wherein the garment is configured to increase nitric oxide levels in a wearer's body.

Embodiment 17. The article of one of Embodiments 15 or 16, wherein the garment is a member selected from the group consisting of shirts, undergarments, socks, leggings, and tights.

Embodiment 18. The article of Embodiment 17, wherein the garment is a shirt.

Embodiment 19. The article of one of Embodiments 17 or 18, wherein the shirt is a short-sleeved shirt.

Embodiment 20. The article of one of Embodiments 17 or 18, wherein the shirt is a long-sleeved shirt.

Embodiment 21. A method of forming a thermoregulating therapeutic yarn comprising:
forming a yarn structure from a first plurality of fibers and a second plurality of fibers, the second fiber comprising a carbon material;
applying a bonding agent mixture to the yarn structure, the bonding agent mixture comprising a bonding agent and nanoparticles, wherein the nanoparticles are capable of upconverting incident wavelengths; and
curing the bonding agent mixture to the yarn structure to form the thermoregulating therapeutic yarn.

Embodiment 22. The method of Embodiment 20, wherein about 15 wt % to about 25 wt % of the thermoregulating therapeutic yarn comprises the carbon material.

Embodiment 23. The method of one of Embodiments 21 or 22, wherein the carbon material comprises carbon fibers.

Embodiment 24. The method of any one of Embodiments 21 to 23, wherein the nanoparticles are present in an amount of from about 1 wt % to about 2 wt % of the thermoregulating therapeutic yarn.

Embodiment 25. The method of any one of Embodiments 21 to 24, wherein the nanoparticles are suspended within the cured bonding agent mixture.

Embodiment 26. The method of any one of Embodiments 21 to 25, further comprising applying a lubricant to the yarn structure.

Embodiment 27. The method of Embodiment 26, wherein the lubricant is applied before the bonding agent mixture.

Embodiment 28. The method of Embodiment 26, wherein the bonding agent mixture comprises the lubricant such that the lubricant is applied simultaneously with the bonding agent and nanoparticles.

Embodiment 29. The method of any one of Embodiments 21 to 28, wherein the second fiber is arranged on the outer surfaces of the yarn structure.

Embodiment 30. The method of any one of Embodiments 21 to 29, wherein the yarn structure is formed as a composite yarn, wherein the first plurality of fibers is a core yarn of the composite yarn, and wherein the second plurality of fibers is a cover yarn of the composite yarn.

Embodiment 31. The method of any one of Embodiments 21 to 29, wherein the yarn structure is formed as a blended yard, and wherein the first and second pluralities of fibers are comingled.

Embodiment 32. The method of any one of Embodiments 21 to 30, wherein the nanoparticles comprise silica.

Embodiment 33. The method of any one of Embodiments 21 to 32, further comprising applying dye molecules to the yarn structure, the dye molecules being configured to produce reactive oxygen species when activated by visible light.

Embodiment 34. A method of forming a thermoregulating therapeutic yarn comprising:
applying a bonding agent mixture to a first plurality of fibers, the bonding agent mixture comprising a bonding agent and nanoparticles;
curing the bonding agent mixture to the first plurality of fibers; and
forming a yarn structure from the first plurality of fibers comprising the cured bonding agent mixture and from a second plurality of fibers to form the thermoregulating therapeutic yarn, wherein the first plurality of fibers or the second plurality of fibers comprises a carbon material.

Embodiment 35. The method of Embodiment 34, wherein about 15 wt % to about 25 wt % of the thermoregulating therapeutic yarn comprises the carbon material.

Embodiment 36. The method of one of Embodiments 34 or 35, wherein the nanoparticles are present in an amount of from about 1 wt % to about 2 wt % of the thermoregulating therapeutic yarn.

Embodiment 37. The method of any one of Embodiments 34 to 36, wherein the nanoparticles are suspended within the cured bonding agent mixture.

Embodiment 38. The method of any one of Embodiments 34 to 36, further comprising applying a lubricant to the first plurality of fibers.

Embodiment 39. The method of Embodiment 38, wherein the lubricant is applied to the first plurality of fibers before the bonding agent mixture.

Embodiment 40. The method of Embodiment 38, wherein the bonding agent mixture comprises the lubricant such that the lubricant is applied to the first plurality of fibers simultaneously with the bonding agent and nanoparticles.

Embodiment 41. The method of any one of Embodiments 34 to 40, further comprising applying the bonding agent mixture to the second plurality of fibers; and curing the bonding agent mixture to the second plurality of fibers.

Embodiment 42. The method of Embodiment 41, further comprising applying a lubricant to the second plurality of fibers.

Embodiment 43. The method of Embodiment 42, wherein the lubricant is applied to the second plurality of fibers before the bonding agent mixture.

Embodiment 44. The method of Embodiment 42, wherein the bonding agent mixture comprises the lubricant such that the lubricant is applied to the second plurality of fibers simultaneously with the bonding agent and nanoparticles.

Embodiment 45. The method of any one of Embodiments 34 to 44, wherein the second plurality of fibers is arranged on the outer surfaces of the yarn structure.

Embodiment 46. The method of any one of Embodiments 34 to 45, wherein the yarn structure is formed as a composite yarn, wherein the first plurality of fibers is a core yarn of the composite yarn, and wherein the second plurality of fibers is a cover yarn of the composite yarn.

Embodiment 47. The method of any one of Embodiments 34 to 45, wherein the yarn structure is formed as a blended yard, and wherein the first and second pluralities of fibers are comingled.

Embodiment 48. The method of any one of Embodiments 34 to 47, wherein the nanoparticles comprise silica.

Embodiment 49. The method of any one of Embodiments 34 to 48, further comprising applying dye molecules to the yarn structure, the dye molecules being configured to produce reactive oxygen species when activated by visible light.

Embodiment 50. The method of any one of Embodiments 34 to 49, further comprising applying dye molecules to the first plurality of fibers, the dye molecules being configured to produce reactive oxygen species when activated by visible light.

Embodiment 51. The method of any one of Embodiments 34 to 50, further comprising applying dye molecules to the second plurality of fibers, the dye molecules being configured to produce reactive oxygen species when activated by visible light.

Embodiment 52. The method of any one of Embodiments 34 to 51, wherein the carbon material comprises carbon fibers.

Obviously, numerous modifications and variations of the present invention are possible in light of the above teachings. It is therefore to be understood that within the scope of the appended claims, the invention may be practiced otherwise than as specifically described herein.

1. A thermoregulating therapeutic yarn comprising:
a first plurality of fibers;
a second plurality of fibers comprising a carbon material, wherein the first and second pluralities of fibers together form a yarn structure; and
nanoparticles adhered to outer surfaces of the first plurality of fibers, second plurality of fibers, or the yarn structure formed therefrom, wherein the nanoparticles are capable of upconverting incident wavelengths.

2. The thermoregulating therapeutic yarn of claim 1, wherein about 15 wt % to about 25 wt % of the thermoregulating therapeutic yarn comprises the carbon material.

3. The thermoregulating therapeutic yarn of claim 1, wherein the nanoparticles are present in an amount of from about 1 wt % to about 2 wt % of the thermoregulating therapeutic yarn.

4. The thermoregulating therapeutic yarn of claim 1, further comprising a bonding agent.

5. The thermoregulating therapeutic yarn of claim 4, wherein the nanoparticles are suspended within the bonding agent.

6. The thermoregulating therapeutic yarn of claim 4, further comprising a lubricant, wherein the lubricant is arranged between the bonding agent and the first and second pluralities of fibers.

7. The thermoregulating therapeutic yarn of claim 1, wherein the second plurality of fibers is arranged on the outer surfaces of the yarn structure.

8. The thermoregulating therapeutic yarn of claim 1, wherein the yarn structure is a composite yarn, the first plurality of fibers is a core yarn of the composite yarn, and the second plurality of fibers is a cover yarn of the composite yarn.

9. The thermoregulating therapeutic yarn of claim 1, wherein the yarn structure is a blended yard, wherein the first and second pluralities of fibers are comingled.

10. The thermoregulating therapeutic yarn of claim 1, wherein the nanoparticles comprise silica.

11. The thermoregulating therapeutic yarn of claim 1, wherein the carbon material comprises carbon fibers.

12. The thermoregulating therapeutic yarn of claim 1, further comprising dye molecules configured to produce reactive oxygen species when activated by visible light.

13. An article prepared from the thermoregulating therapeutic yarn of claim 1.

14. The article of claim 13, wherein the article is a member selected from bed sheets, pillowcases, and bandages.

15. The article of claim 13, wherein the article is a garment.

16. The article of claim 15, wherein the garment is configured to increase nitric oxide levels in a wearer's body.

17. The article of claim 15, wherein the garment is a member selected from the group consisting of shirts, undergarments, socks, leggings, and tights.

18. The article of claim 17, wherein the garment is a shirt.

19. The article of claim 17, wherein the shirt is a short-sleeved shirt.

20. The article of claim 17, wherein the shirt is a long-sleeved shirt.

21. A method of forming a thermoregulating therapeutic yarn comprising:
forming a yarn structure from a first plurality of fibers and a second plurality of fibers, the second fiber comprising a carbon material;
applying a bonding agent mixture to the yarn structure, the bonding agent mixture comprising a bonding agent and nanoparticles, wherein the nanoparticles are capable of upconverting incident wavelengths; and
curing the bonding agent mixture to the yarn structure to form the thermoregulating therapeutic yarn.

22. The method of claim 21, wherein about 15 wt % to about 25 wt % of the thermoregulating therapeutic yarn comprises the carbon material.

23. The method of claim 21, wherein the carbon material comprises carbon fibers.

24. The method of claim 21, wherein the nanoparticles are present in an amount of from about 1 wt % to about 2 wt % of the thermoregulating therapeutic yarn.

25. The method of claim 21, wherein the nanoparticles are suspended within the cured bonding agent mixture.

26. The method of claim 21, further comprising applying a lubricant to the yarn structure.

27. The method of claim 26, wherein the lubricant is applied before the bonding agent mixture.

28. The method of claim 26, wherein the bonding agent mixture comprises the lubricant such that the lubricant is applied simultaneously with the bonding agent and nanoparticles.

29. The method of claim 21, wherein the second fiber is arranged on the outer surfaces of the yarn structure.

30. The method of claim 21, wherein the yarn structure is formed as a composite yarn, wherein the first plurality of fibers is a core yarn of the composite yarn, and wherein the second plurality of fibers is a cover yarn of the composite yarn.

31. The method of claim 21, wherein the yarn structure is formed as a blended yard, and wherein the first and second pluralities of fibers are comingled.

32. The method of claim 21, wherein the nanoparticles comprise silica.

33. The method of claim 21, further comprising applying dye molecules to the yarn structure, the dye molecules being configured to produce reactive oxygen species when activated by visible light.

34. A method of forming a thermoregulating therapeutic yarn comprising:
applying a bonding agent mixture to a first plurality of fibers, the bonding agent mixture comprising a bonding agent and nanoparticles;
curing the bonding agent mixture to the first plurality of fibers; and
forming a yarn structure from the first plurality of fibers comprising the cured bonding agent mixture and from a second plurality of fibers to form the thermoregulating therapeutic yarn, wherein the first plurality of fibers or the second plurality of fibers comprises a carbon material.

35. The method of claim 34, wherein about 15 wt % to about 25 wt % of the thermoregulating therapeutic yarn comprises the carbon material.

36. The method of claim 34, wherein the nanoparticles are present in an amount of from about 1 wt % to about 2 wt % of the thermoregulating therapeutic yarn.

37. The method of claim 34, wherein the nanoparticles are suspended within the cured bonding agent mixture.

38. The method of claim 34, further comprising applying a lubricant to the first plurality of fibers.

39. The method of claim 38, wherein the lubricant is applied to the first plurality of fibers before the bonding agent mixture.

40. The method of claim 38, wherein the bonding agent mixture comprises the lubricant such that the lubricant is applied to the first plurality of fibers simultaneously with the bonding agent and nanoparticles.

41. The method of claim 34, further comprising applying the bonding agent mixture to the second plurality of fibers; and curing the bonding agent mixture to the second plurality of fibers.

42. The method of claim 41, further comprising applying a lubricant to the second plurality of fibers.

43. The method of claim 42, wherein the lubricant is applied to the second plurality of fibers before the bonding agent mixture.

44. The method of claim 42, wherein the bonding agent mixture comprises the lubricant such that the lubricant is applied to the second plurality of fibers simultaneously with the bonding agent and nanoparticles.

45. The method of claim 34, wherein the second plurality of fibers is arranged on the outer surfaces of the yarn structure.

46. The method of claim 34, wherein the yarn structure is formed as a composite yarn, wherein the first plurality of fibers is a core yarn of the composite yarn, and wherein the second plurality of fibers is a cover yarn of the composite yarn.

47. The method of claim 34, wherein the yarn structure is formed as a blended yard, and wherein the first and second pluralities of fibers are comingled.

48. The method of claim 34, wherein the nanoparticles comprise silica.

49. The method of claim 34, further comprising applying dye molecules to the yarn structure, the dye molecules being configured to produce reactive oxygen species when activated by visible light.

50. The method of claim 34, further comprising applying dye molecules to the first plurality of fibers, the dye molecules being configured to produce reactive oxygen species when activated by visible light.

51. The method of claim 34, further comprising applying dye molecules to the second plurality of fibers, the dye molecules being configured to produce reactive oxygen species when activated by visible light.

52. The method of claim 34, wherein the carbon material comprises carbon fibers.
